(19) United States
(12) Patent Application Publication
(10) Pub. No.: US 2013/0053711 A1
(43) Pub. Date: Feb. 28, 2013

(54) Implantable Device for Detecting Variation in Fluid Flow Rate

(71) Applicants: Rama Krishna Kotlanka, Singapore (SG); Pradeep Basappa Khannur, Singapore (SG); Kok Lim Chan, Singapore (SG); Soo Yeng Benjamin Chua, Singapore (SG); Xiaojun Yuan, Singapore (SG); Minkyu Je, Singapore (SG); Vaidyanathan Kripesh, Singapore (SG); Daquan Yu, Singapore (SG); Pavel Neuzil, Singapore (SG); Lichun Shao, Singapore (SG); Woo Tae Park, Singapore (SG)

(72) Inventors: Rama Krishna Kotlanka, Singapore (SG); Pradeep Basappa Khannur, Singapore (SG); Kok Lim Chan, Singapore (SG); Soo Yeng Benjamin Chua, Singapore (SG); Xiaojun Yuan, Singapore (SG); Minkyu Je, Singapore (SG); Vaidyanathan Kripesh, Singapore (SG); Daquan Yu, Singapore (SG); Pavel Neuzil, Singapore (SG); Lichun Shao, Singapore (SG); Woo Tae Park, Singapore (SG)

(21) Appl. No.: 13/503,954

(22) PCT Filed: Sep. 27, 2010
(86) PCT No.: PCT/SG2010/000363

§ 371 Date: Nov. 9, 2012

(30) Foreign Application Priority Data
Oct. 30, 2009 (SG) 2009072448

(51) Int. Cl. A61B 5/0265

(52) U.S. Cl. 600/505

(57) ABSTRACT

According to embodiments of the present invention, an implantable device for detecting variation in fluid flow rate is provided. The implantable device includes: a substrate having an active element arrangement; a sensor arrangement having a first portion that is mechanically secured and a second portion that is freely deflectable, the sensor arrangement in electrical communication with the active element arrangement, wherein the active element arrangement is configured to detect changes in deformation of the sensor arrangement and produce an output in response to the detected changes; and at least one inductive element mechanically coupled to the substrate and in electrical communication with the active element arrangement, wherein the inductive element is adapted to power the active element arrangement through inductive coupling to an excitation source, and wherein the inductive element is adapted to transmit the output associated with the detected changes in the sensor.

### TECHNICAL FIELD

Various embodiments relate to an implantable device for detecting variation in fluid flow rate.

### BACKGROUND

Prosthetic grafts are frequently used in vascular surgery in the context of bypass surgery for lower limb ischemia or as a conduit for haemodialysis in renal failure.

In these settings, graft failure can result in deleterious outcomes for patients, such as worsening ischemia and inability to undergo haemodialysis.

Insufficient blood flow rates in these grafts are predictive of subsequent graft thrombosis and failure. Underlying this is the presence of stenoses in the graft or downstream from the graft. Variations in flow rates can localize the position of significant stenoses that may result in graft thrombosis.

Flow rate monitoring provides an indication for early intervention to prevent graft failure. Accordingly, literature devoted to detecting failing grafts viz-a-viz decreasing flow rates exists. For instance, there are systems using different modalities to monitor graft flow rates and detect early failures. These modalities include ultrasound, computer tomography (CT) scan and formal angiograms. The disadvantages of these modalities include the need for significant amounts of procedural time (ultrasound, angiogram) and the use of nephrotoxic contrast (CT scan and angiograms). These procedures are not entirely risk-free for patients and come with some procedural morbidity.

Commercially available flow rate detection devices also exist. These devices require specific user training, bulky machine attachments and significant financial costs. An example of such a device is made by the Transonic® company. Such systems are not implantable.

There is thus a need to provide a system that can provide continuous monitoring of blood flow in vascular prosthetic grafts using simple hand held devices.

### SUMMARY

According to an embodiment, an implantable device for detecting variation in fluid flow rate is provided. The implantable device may include: a substrate having an active element arrangement; a sensor arrangement having a first portion that is mechanically secured and a second portion that is freely deflectable, the sensor arrangement in electrical communication with the active element arrangement, wherein the active element arrangement is configured to detect changes in deformation of the sensor arrangement and produce an output, in response to the detected changes; and at least one inductive element mechanically coupled to the substrate and in electrical communication with the active element arrangement, wherein the inductive element is adapted to power the active element arrangement through inductive coupling to an excitation source, and wherein the inductive element is adapted to transmit the output associated with the detected changes in the sensor arrangement.

Various embodiments may provide for a highly sensitive, ultra low power implantable sensor that is interrogated through wireless means placed within a graft. The implantable device can be implemented in prosthetic grafts used in vascular interventions. Similarly, it can be used in vascular vessels in organ transplants. The implantable device is usable as a flow meter or pressure sensor for biomedical applications both in-vivo and in-vitro.

In the context of various embodiments, the term “implantable device” may mean a device to be implanted or internally located within an organism. The implantable device may be responsive to an interrogation circuit having an exciter/interrogator element which is located outside the organism. The implantable device has a structure implantable within the organism and is operatively configured to carry out or assist in carrying out a function (such as monitoring a health parameter such as physiological parameters like blood flow, pressure and temperature) within the organism.

In the context of various embodiments, the term “variation” may mean that the implantable device may be configured to not only sense changes in a fluid flow rate, but also to detect that a fluid is flowing at where the implantable device is located. In the context of various embodiments, the term “fluid” may mean a liquid (such as water, blood, plasma) or a gas.

The term “substrate” may be understood in the context of semiconductor technology, i.e. “substrate” may refer to bulk semiconductor material forming a base material for fabricating electronics thereon or therein or for growing further layers of semiconductor material thereon. The term “active element arrangement” may mean one or more elements, fabricated as integrated electronics thereon or therein the substrate, that require power to work. Examples of the one or more elements may include devices that provide processing functions such as AND, NAND, or OR logic using transistors, resistors, capacitors, inductors and the like. Each of the one or more elements may serve any purpose, for example as a data transmitter.

In the context of various embodiments, the term “sensor arrangement” may mean a micro or nano-sized sensing element having at least one portion that is movable or deformable, so that actuation of the movable portion or deformation of the deformable portion changes the electrical properties of the sensing element. In other embodiments, the sensor arrangement may mean a movable structure having at least one portion that is movable or deformable, the movable structure provided with at least one sensing element. The change in the electrical properties may include a change in the resistance of the sensing element. In various embodiments, the first portion of the sensor arrangement may be mechanically secured to any portion of the substrate. In other embodiments, the first portion of the sensor arrangement may be mechanically secured to any portion of the at least one inductive element.

In various embodiments, the term “freely deflectable” may mean that the second portion of the sensor arrangement experiences a degree of movement in the presence of fluid flow, which may bring about a change in shape of the sensor arrangement. The degree of movement may be such that the second portion of the sensor arrangement pivots about the first portion of the sensor arrangement that is mechanically secured; or may be centered about the second portion itself so that the first portion remains stationary while the second portion moves.

The term “configured” may mean that the active element arrangement is provided with electronics that are designed to measure changes in deformation of the sensor arrangement.

In the context of various embodiments, the term “deformation” may mean a change in the shape or size of the sensor arrangement due to fluid flow past the sensor arrangement. The deformation may occur at any portion of the sensor arrangement, although it typically occurs at the second portion of the sensor arrangement (since the second portion is freely deflectable). A change in the shape or size of the sensor arrangement may occur from portions of the sensor arrangement being displaced, from a position at rest, due to the fluid flow. For instance, a change in the shape of the sensor arrangement may occur when only one portion of the sensor arrangement moves, while the remainder of the sensor arrangement remains in its original position.

In the context of various embodiments, the term “inductive element” may mean any device that allows coupling to a magnetic field, the inductive element converting the magnetic energy to electrical energy that is able to power the active element arrangement of the substrate. The magnetic field may be externally generated, i.e. not from the implantable device itself. The inductive element may have a shape not limited to that of a helix or a coil.

In various embodiments, the term “excitation source” may mean an external means (such as another inductor coupled to external circuitry) capable of powering the active element arrangement through induction. The excitation source may not be located within the substrate itself, but may be provided as a separate circuit arrangement. While in various embodiments it may be provided to have the separate circuit arrangement located outside the organism, the separate circuit arrangement may also be located internally of the organism, but at a different place from where the implantable device is located.

In various embodiments, the sensor arrangement may include a movable structure; and at least one sensing element formed on or within the movable structure or an anchor of the movable structure. In the context of various embodiments, the term “movable structure” may mean a structure having a deformable nature, which may be flexibly resilient. In the context of various embodiments, the term “formed on” may mean that the sensing element is provided at a surface of the movable structure, while the term “within” may mean that the sensing element is embedded inside the movable structure. In the context of various embodiments, the term “sensing element” may mean the portion of the implantable device from which changes of electrical properties, such as piezoresistance, are measured to detect variation in fluid flow rate. The changes of the electrical properties may be brought about by stress arising from the sensing element being deformed, such as from deflection of the movable structure.

In various embodiments, the movable structure may include a cantilever or diaphragm providing anchorage to the sensing element. In various embodiments, the cantilever may be a beam supported at only one end.

In various embodiments, the at least one sensing element may include one or more of the following structures: a nanowire, a piezoresistor, a capacitor, a piezoelectric transducer or a resonator.

According to an embodiment, an implantable device for detecting variation in fluid flow rate is provided. The implantable device may include: a first sensor configured to detect a first pressure; a second sensor disposed downstream of the first sensor and configured to detect a second pressure; a housing structure having an active element arrangement, both the first sensor and the second sensor being in electrical communication with the active element arrangement, wherein the active element arrangement is configured to obtain the first pressure, the second pressure and produce an output containing information on the difference between the first pressure and the second pressure; and at least one inductive element mechanically coupled to the housing structure and in electrical communication with the active element arrangement, wherein the inductive element is adapted to power the active element arrangement through inductive coupling to an excitation source, and wherein the inductive element is adapted to transmit the output from the active element arrangement.

In the context of various embodiments, the term “sensor” may mean a device capable of measuring pressure, the device being micro or nano-sized. The device may be sensitive to pressure in that its electrical properties change when subject to different pressures. The change in the electrical properties may include a change in the resistance of the sensor. In various embodiments, the first sensor and/or the second sensor may be mechanically secured to any portion of the substrate. In other embodiments, the housing structure may be mechanically secured to any portion of the at least one inductive element. In the context of various embodiments, the term “first sensor and/or the second sensor” may mean either the first sensor, the second sensor or both.

In the context of various embodiments, the term “downstream” may mean the first and the second detectors are spaced apart, within the implantable device, along the direction flow of the fluid being detected.

In various embodiments, the term “housing structure” may mean any structure, such as a substrate fabricated from semiconductor material, upon which the first sensor, the second sensor and the active element arrangement are provided. In one embodiment, the first sensor, the second sensor and the active element arrangement are fabricated directly onto the housing structure. In another embodiment, the first sensor, the second sensor and the active element arrangement may be mounted onto the housing structure.

In various embodiments, the active element arrangement may include application specific integrated circuitry (ASIC). The application specific integrated circuitry may include a sensor interface coupled to the sensor arrangement; or the sensor interface may be coupled to the first sensor or the second sensor. The application specific integrated circuitry may further include an analog to digital data converter coupled to the sensor interface, the analog to digital data converter converting the output from the sensor interface, such as the output associated with the detected changes in the sensing element of the sensor arrangement into digital data or the output containing information on the difference between the two pressures detected by the first sensor and the second sensor. The application specific integrated circuitry may further include a data modulator coupled to the analog to digital data converter and the at least one inductive element, the data modulator sending the digital data to the at least one inductive element for transmission. The application specific integrated circuitry may further include a clock device coupled to synchronise the operation of the amplifier, the analog to digital data converter and the data modulator. The application specific integrated circuitry may further include an energy management device coupled to the at least one inductive element, to power the clock device, the amplifier, the analog to digital data converter and the data modulator.

In various embodiments, the sensor arrangement; the first sensor and/or the second sensor may be a nano electromechanical structure or a micro electromechanical structure. In the context of various embodiments, the term “electromechanical” may mean that the sensor arrangement; the first sensor and/or the second sensor are such that their electrical properties (such as resistance) may be changed when the sensor arrangement; the first sensor and/or the second sensor are subjected to mechanical forces that may alter the shape of or actuate the sensor arrangement; the first sensor and/or the second sensor.

The first sensor and/or the second sensor may include one or more of the following sensing element: a nanowire, a piezoresistor, a capacitor, a piezoelectric sensor, or a resonator. The sensor arrangement; the first sensor and/or the second sensor may be disposed between the at least one inductive element and a further element formed of resilient material providing anchorage when deformed. The sensor arrangement; the first sensor and/or the second sensor may be adapted to measure variation in blood flow rate.

In various embodiments, the at least one inductive element may be formed of resilient material providing anchorage when the inductive element is deformed. The at least one inductive element may be a coil. The at least one inductive element may be formed of nitinol or titanium.

In various embodiments, the housing structure may be formed of bulk silicon, silicon oxide or polymer.

In various embodiments, a vessel may be provided. The vessel may have an interior surface to which an implantable device, built in accordance with various embodiments, is secured through biasing engagement by the at least one inductive element of the implantable device. In the context of various embodiments, the term “vessel” may mean any hollow structure, such as a tube or pipe, that is open on opposite ends and allows fluid to pass through the hollow structure.

In various embodiments, the vessel may have an interior surface to which an implantable device, built in accordance with various embodiments, is embedded. The vessel may be used as a prosthethic graft.

In various embodiments, a method of placing an implantable device into the interior of a vessel is provided. The method may include providing a guide rod with an implantable device, built in accordance with various embodiments; inserting the guide rod into the interior of a vessel; and releasing the at least one inductive element of the implantable device to bias against the interior of the vessel so that the implantable device is secured to the interior of the vessel.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1A is a perspective view of an implantable device built according to one embodiment.

FIG. 1B is a perspective view of a sensor arrangement of the implantable device of FIG. 1A.

FIG. 1C is a perspective view of an implantable device built according to one embodiment of the present invention.

FIG. 2 shows a schematic of application specific integrated circuitry, being part of an active element arrangement present within an implantable device, according to one embodiment.

FIG. 3 shows a flow chart illustrating a method of placing an implantable device, according to various embodiments, into the interior of a vessel.

FIGS. 4A to 4C illustrate one implementation of the flow chart of FIG. 3.

FIG. 5 shows a prosthetic vascular graft that is implanted within an organism.

FIG. 6 shows a cross-sectional view of a portion of the housing structure of the implantable device of FIG. 1C.

FIGS. 7A to 7F show cross-sectional views of another fabrication process to manufacture a sensor of an implantable device, in accordance with various embodiments.

FIGS. 8A to 8C show cross-sectional views of an assembly process to produce an implantable device in accordance with various embodiments.

FIG. 9 shows chip architecture for an ASIC used by an implantable device according to various embodiments.

FIG. 10 shows a schematic diagram of a RF front-end of an ASIC used by an implantable device according to various embodiments.

FIG. 11 shows a flow chart implemented in a digital baseband and controller of an ASIC used by an implantable device according to various embodiments.

FIG. 12A shows a schematic diagram of a sensor interface circuit of an ASIC used by an implantable device according to various embodiments.

FIG. 12B shows a timing diagram to illustrate operation of the sensor interface circuit of FIG. 12A.

FIG. 13 shows a schematic diagram of a SAR ADC of an ASIC used by an implantable device according to various embodiments.

FIG. 14 shows a common-mode resetting tri-level switching scheme applied to the SAR ADC of an ASIC used by an implantable device according to various embodiments.

FIG. 15 shows a microphotograph of an ASIC chip by an implantable device according to various embodiments.

FIG. 16 shows measure waveforms in the ASIC chip of FIG. 15.

FIG. 17 shows measured timing diagrams.

FIGS. 18A to 18D show experiments conducted, for detecting fluid velocity, using an implantable device, according to various embodiments.

FIG. 18E shows data on fluid flow in a nanosensor shown in FIG. 18F.

FIG. 18G shows drag force on a sensor of an implantable device, according to an embodiment.

FIG. 19A shows a fabricated sensor, according to an embodiment.

FIG. 19B shows deformation of a cantilever of an implantable device, according to an embodiment.

FIG. 19C shows a schematic of an experiment conducted using an implantable device, according to an embodiment.

FIG. 19D plots results of sensor response.

FIG. 20A shows a plot of parameters for sensors of an implantable device, according to an embodiment.

FIG. 20B shows a stenoses graft having 50% blockage.

FIG. 21 shows a schematic of an 8-bit successive-approximation ADC register.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

According to various embodiments of the invention, a Micro-Electro-Mechanical Systems (MEMS)/Nano-Electro-Mechanical Systems (NEMS) based flow sensor is disclosed. The MEMS/NEMS sensor is an ultralow power (<10uW) IC with a wireless interface. The MEMS/NEMS sensor may be integrated along with the graft manufacturing or it may be placed in a vessel, wherein a coil of the MEMS/NEMS sensor acts as an anchor. The sensor may be subjected to drag force (and lift) due to blood flow and thus a diaphragm/cantilever sensing element of the MEMS/NEMS sensor deforms. The deformation intensity may depend on several factors, such as the flow velocity of the blood and the dimensions of the diaphragm/cantilever sensing element, which affect the stiffness of the diaphragm/cantilever sensing element. This deformation induces a change in the electrical performance of the diaphragm/cantilever sensing element, which will be picked by application specific integrated circuitry (ASIC) on the MEMS/NEMS sensor. Powering of the ASIC and data communication with the same is through wireless means (RF), i.e. by inductive coupling. All primary components of the NEMS/MEMS, ASIC, diaphragm/cantilever sensing element and anchor coil will be provided in a biocompatible package.

In various embodiments, the coil provides not only an anchor, but also facilitates inductive energy transfer to power the ASIC and also for transmission of data from the ASIC. The sensor has a deformable/movable structure which will be deflected due to the drag of fluid flow, which causes changes in electrical performance of the sensor. Being movable, the sensor has the advantage of less likely causing thrombosis (just like a mechanical heart valve).

Various embodiments may provide for implantable microsystems, having ultra miniaturized sensor, ultra low power ICs and a wireless interface, for continuous monitoring of blood flow.

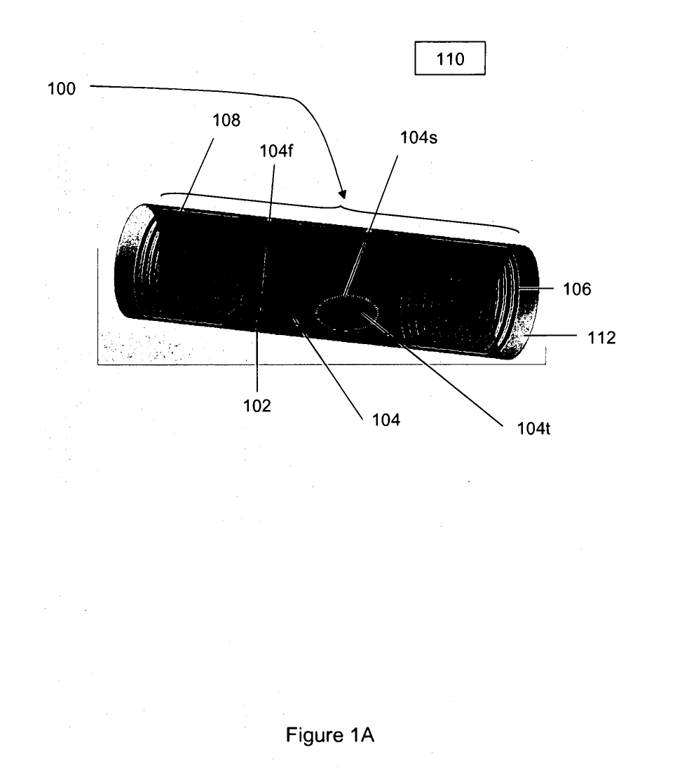

FIG. 1A is a perspective view of an implantable device 100 built according to one embodiment of the present invention. The implantable device 100 is suitable for detecting variation in fluid flow rate.

The implantable device 100 has a substrate 102 having an active element arrangement; a sensor arrangement 104 having a first portion 104f that is mechanically secured and a second portion 104s that is freely deflectable. The sensor arrangement 104 is in electrical communication with the active element arrangement, wherein the active element arrangement is configured to detect changes in deformation of the sensor 104 and produce an output in response to the detected changes. At least one inductive element 106, 108 is mechanically coupled to the substrate 102 and in electrical communication with the active element arrangement, wherein the inductive element 106, 108 is adapted to power the active element arrangement through inductive coupling to an excitation source 110. The inductive element 106, 108 is adapted to transmit the output associated with the detected changes in the sensor arrangement 104.

In the embodiment shown in FIG. 1A, the implantable device 100 has two inductive elements 106, 108. While FIG. 1A shows that each of the two inductive elements 106, 108 is shaped like a coil, other shapes are possible. While both inductive elements 106, 108 may be in electrical communication with the active element arrangement inside the substrate 102 to power the active element arrangement, it is sufficient to have a first of the inductive elements (either 106 or 108) be in electrical communication with and power the active element arrangement. Accordingly, a second of the inductive elements acts as a dummy coil, i.e. a further element formed of resilient material providing anchorage to a vessel 112 to which the implantable device 100 is attached, when the second of the inductive elements is deformed. Thus, in another embodiment of the present invention (not shown), the implantable device may have the following configuration: a substrate with an active element arrangement; a sensor having a first portion that is mechanically secured and a second portion that is freely deflectable, the sensor being in electrical communication with the active element arrangement. The active element arrangement is configured to detect changes in deformation of the sensor and produce an output in response to the detected changes. One inductive element is mechanically coupled to the substrate and in electrical communication with the active element arrangement, wherein the inductive element is adapted to power the active element arrangement through inductive coupling to an excitation source. The inductive element is adapted to transmit the output associated with the detected changes in the sensor.

Returning to FIG. 1A, the sensor arrangement 104 is a micro-sized or nano-sized structure having at least one portion that is movable or deformable, so that actuation of the movable portion or deformation of the deformable portion changes the electrical properties of the sensor arrangement 104. The deformation may occur at any portion of the sensor arrangement 104, although it typically occurs at the second portion 104s of the sensor arrangement 104 (since the second portion 104s is freely deflectable). A change in the shape or size of the sensor arrangement 104 may occur from portions of the sensor arrangement 104 being displaced, from a position at rest, due to the fluid flow. In the embodiment shown in FIG. 1A, as fluid, such as a liquid (e.g. water, blood, plasma) or a gas, flows past the sensor arrangement 104, drag force will be exerted on the sensor arrangement 104. The drag force will actuate the second portion 104s of the sensor arrangement 104, thereby varying the height, which the tip 104t of the sensor arrangement 104 protrudes, from the substrate 102 surface. The degree of variation depends on the flow rate, thus making the implantable device 100 sensitive to changes in the fluid flow rate and also able to detect whether a fluid is flowing at where the implantable device is located.

FIG. 1B is a perspective view of the sensor arrangement 104 of the implantable device 100 of FIG. 1A. As mentioned above, the sensor arrangement 104 is a nano electromechanical structure or a micron electromechanical structure.

The sensor arrangement 104 includes a movable structure 120; and at least one sensing element 122 formed on the movable structure 120. In the embodiment shown in FIG. 1B, the sensing element 122 is provided at a surface of the movable structure. In another embodiment (not shown), it will be appreciated that the sensing element 122 is embedded inside the movable structure.

In the embodiment shown in FIG. 1B, the movable structure 120 is a cantilever 126. A first portion 126f of the movable structure 120 serves as an anchor for the cantilever 126 such that the mechanically deformable cantilever 126 is free to deflect, especially at a second end 126s, so as to translate stress onto the sensing element 122.

In the embodiment shown in FIG. 1B, the sensing element 122 is a nanowire 124. The nanowire 124 may be positioned at any suitable position on or within the mechanically deformable cantilever 126, for example at a location where the stress is most effectively being translated thereupon. While the stress arises from the nanowire 124 being deformed (for instance by moving) from the deflection of the second end 126s of the cantilever 126, the stress may also be a tensile stress or a compressive stress. Further, the stress may be a longitudinal stress, a transverse stress or a shear stress with respect to the nanowire 124.

Mechanical stress applied on the nanowire 124 is observed to cause a change in its electrical properties, such as resistance.

In more detail, the cantilever 126 may provide a conductive substrate 126b forming a gate terminal of the nanowire 124. One end 124s of the nanowire 124 may form a source terminal of the nanowire 124 and be connected to a source pad (not shown). Another end 124d of the nanowire 124 positioned opposite to the end 124s may form a drain terminal of the nanowire resistor 124 and be connected to a drain pad (not shown). A bias voltage source (not shown) connected between the conductive substrate 126b and one end 124s of the nanowire 124 may be termed “V_{GS}”. A further bias voltage source (not shown) connected between the end 124s and the end 124d of the nanowire 124. The further bias voltage source may be termed “V_{DS}”.

Giant piezoresistance in the nanowire 124 may be demonstrated by the modulation of an electric field induced with the external electrical bias described above. Positive bias for a p-type device (negative bias for an n-type device) partially depletes the nanowire 124 forming a pinch-off region or depletion region, which resembles a tunnel through which an electrical current is squeezed. This pinch-off region determines the total current flowing through the nanowire 124. At this point, a combination of the electrical biasing and application of the mechanical stress (as outlined above) impacts the charge carrier concentration and mobility, to achieve an electrically controlled giant piezoresistance in the nanowire 124. The phenomenon creates a stress-gated FET, exhibiting a maximum gauge factor (gauge factor is the relative change in electrical resistance per unit mechanical strain) of about 5000, 2 orders of magnitude (from 50 to 5000) increase over bulk value. The application of stress alone may not change the giant piezoresistive coefficient, but it may only change the resistance by changing the concentration and mobility of the charge carriers.

It will be appreciated that, in place of the nanowire 124, other structures (not shown) such as a piezoresistor, a capacitor, a piezoelectric sensor or a resonator are possible for the sensing element 122. A capacitor sensor may be formed by two parallel conductive plates separated by an insulator layer in between. One plate may be fixed and the other may be attached to the movable structure 120, being the cantilever 126 in the embodiment shown in FIG. 1B. The deflection of the cantilever 126 causes relative displacement between the two plates and leads to capacitance change. By measuring the capacitance change, the deflection of the cantilever 126 may be deduced.

Returning to FIG. 1A, the sensor arrangement 104 is disposed between the two inductive elements 106, 108. When one of the two inductive elements serves only to provide anchorage, then the sensor arrangement 104 is disposed between one inductive element and a further element formed of resilient material providing anchorage when deformed. The first portion 104f of the sensor arrangement 104 is mechanically secured, in other words fixed, to the substrate 102. It will be appreciated that in other embodiments of the invention (not shown), the first portion of the sensor arrangement may be mechanically secured, in other words fixed, to any portion of the at least one inductive element.

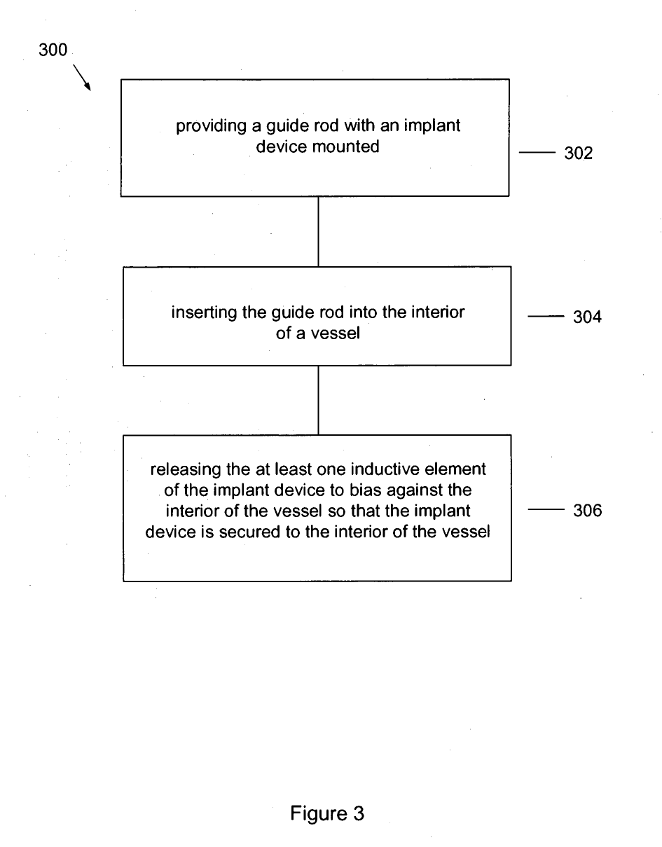

In the embodiment shown in FIG. 1A, the implantable device 100 may be provided inside a vessel 112. The vessel 112 has an interior surface to which an implantable device 100 is secured through biasing engagement by the at least one inductive element (106, 108) of the implantable device 100. Alternatively, the implantable device 100 may be embedded into the interior surface of the vessel 112. The vessel 112 is a hollow structure, open on opposite ends to allow fluid to pass through, that forms part of a prosthethic vascular graft (labeled 502 in FIG. 5) implanted into an organism. It will also be appreciated that, instead of using a prosthethic vascular graft, the implantable device 100 may be surgically inserted into a blood vessel, so that FIG. 1A shows a perspective view of the implantable device 100 being provided inside a blood vessel.

The sensor 104 of the implantable device 100 is adapted to measure variation in blood flow rate within an organism and can therefore facilitate monitoring of a health parameter within the organism. The implantable device 100 may be responsive to an interrogation circuit having exciter/interrogator elements that are in electrical communication with the active element arrangement of the substrate 102. In the embodiment shown in FIG. 1A, the exciter/interrogator elements form part of a separate circuit arrangement, located external to the substrate 102, schematically represented as the excitation source 110.

The excitation source 110 may have an inductor (not shown) capable of powering the active element arrangement of the substrate 102 through induction. The excitation source 110 may be provided with a receiver (which may be another inductor, not shown) that receives the output from the at least one inductive element (106, 108) containing information about detected changes in the sensor 104. The excitation source 110 may also be provided with a processor (not shown) that is coupled to the receiver and processes the information about the detected changes in the sensor 104. The excitation source 110 may then be connected to a suitable interface that can process and display the information about the detected changes in the sensor 104 on a computer screen. It will be appreciated that the receiver and the processor may be placed in another separate circuit arrangement, instead of within the excitation source 110.

FIG. 1C is a perspective view of an implantable device 150 built according to an embodiment. The implantable device 150 is suitable for detecting variation in fluid flow rate.

The implantable device 150 has a housing structure 162 having an active element arrangement 152; a first sensor 154 configured to detect a first pressure; and a second sensor 155 disposed downstream of the first sensor 154 and configured to detect a second pressure. The first sensor 154 and the second sensor 155 are in electrical communication with the active element arrangement 152, wherein the active element arrangement 152 (having application specific integrated circuitry, ASIC) is configured to obtain the first pressure, the second pressure and produce an output containing information on the difference between the first pressure and the second pressure. At least one inductive element 156, 158 is mechanically coupled to the housing structure 162 and in electrical communication with the active element arrangement 152, wherein the inductive element 156, 158 is adapted to power the active element arrangement 152 through inductive coupling to an excitation source 160. The inductive element 156, 158 is adapted to transmit the output from the active element arrangement 152, the output being information on the difference between the first pressure and the second pressure. In the embodiment shown in FIG. 1C, the active element arrangement 152, the first sensor 154, the second sensor 155 and the inductive elements 156, 158 are mechanically coupled to the housing structure 162.

In the embodiment shown in FIG. 1C, the implantable device 150 has two inductive elements 156, 158. While FIG. 1C shows that each of the two inductive elements 156, 158 is shaped like a coil, other shapes are possible. While both inductive elements 156, 158 may be in electrical communication with the active element arrangement 152 provided on the housing structure 162 to power the active element arrangement 152, it is sufficient to have a first of the inductive elements (either 156 or 158) be in electrical communication with and power the active element arrangement 152. Accordingly, a second of the inductive elements acts as a dummy coil, i.e. a further element formed of resilient material providing anchorage to a vessel (not shown) to which the implantable device 150 is attached, when the second of the inductive elements is deformed. Thus, in another embodiment (not shown), the implantable device may have the following configuration: a housing structure with an active element arrangement; a first sensor; and a second sensor disposed downstream of the first sensor, the first sensor and the second sensor being in electrical communication with the active element arrangement. The active element arrangement is configured to obtain the first pressure detected by the first sensor, the second pressure detected by the second sensor and produce an output containing information on the difference between the two pressures detected. One inductive element is mechanically coupled to the housing structure and in electrical communication with the active element arrangement, wherein the inductive element is adapted to power the active element arrangement through inductive coupling to an excitation source. The inductive element is adapted to transmit the output being information on the difference between the two pressures detected.

Returning to FIG. 1C, the first sensor 154 and the second sensor 155 are micro-sized or nano-sized structures, whose electrical properties (such as resistance) change when subject to different pressures.

In the embodiment shown in FIG. 1C, by measuring the pressure difference at the first sensor 154 and the second sensor 155 along fluid [such as a liquid (e.g. water, blood, plasma) or a gas] flow direction, the flow velocity can be indirectly deduced using the Navier-Stokes formula:

−ΔP/Δx=8ηQ/(πRᵢ⁴)+ρ/(πRᵢ²)*dQ/dt

where Q is the flow rate, η is the viscosity, ρ is the density, Rᵢ is the lumen radius, ΔP is the pressure difference and Δx is the distance between the two sensors 154, 155 along the flow direction. By taking measurements of flow velocity at various instances, variation in fluid flow rate may be obtained.

The first sensor 154 and the second sensor 155 are disposed between the two inductive elements 156, 158. When one of the two inductive elements serves only to provide anchorage, then the first sensor 154 and the second sensor 155 are disposed between one inductive element and a further element formed of resilient material providing anchorage when deformed. The first sensor 154 and the second sensor 155 are mechanically secured, in other words fixed, to the housing structure 162. It will be appreciated that in other embodiments of the invention (not shown), either of the first sensor 154 and the second sensor 155 may be mechanically secured, in other words fixed, to any portion of the at least one inductive element.

In the embodiment shown in FIG. 1C, the implantable device 150 may be provided inside a vessel (not shown). The vessel may have an interior surface to which the implantable device 150 is secured through biasing engagement by the at least one inductive element (156, 158) of the implantable device 150. Alternatively, the implantable device 150 may be embedded into the interior surface of the vessel. The vessel may have a hollow structure, open on opposite ends to allow fluid to pass through, that forms part of a prosthetic vascular graft (labeled 502 in FIG. 5) implanted into an organism. It will also be appreciated that, instead of using a prosthetic vascular graft, the implantable device 150 may be surgically inserted into a blood vessel.

The first sensor 154 and the second sensor 155 of the implantable device 150 are adapted to measure variation in blood flow rate within an organism and can therefore facilitate monitoring of a health parameter within the organism. The implantable device 150 may be responsive to an interrogation circuit having exciter/interrogator elements that are in electrical communication with the sensors 154 and 155. In the embodiment shown in FIG. 1C, the exciter/interrogator elements form part of a separate circuit arrangement, located in the housing structure 162.

The excitation source 160 may have an inductor (not shown) capable of powering the active element arrangement 152 through inductive coupling. The excitation source 160 may also include a receiver (which may be another inductor, not shown) that receives the output from the at least one inductive element (156, 158) containing information on the difference between the two pressures detected by the first sensor 154 and the second sensor 155. The excitation source 160 may also be provided with a processor (not shown) that is coupled to the receiver and processes the information on the difference between the two pressures detected. The excitation source 160 may then be connected to a suitable interface that can process and display the information on the difference between the two pressures detected on a computer screen. It will be appreciated that the receiver and the processor may be placed in another separate circuit arrangement, instead of within the excitation source 160.

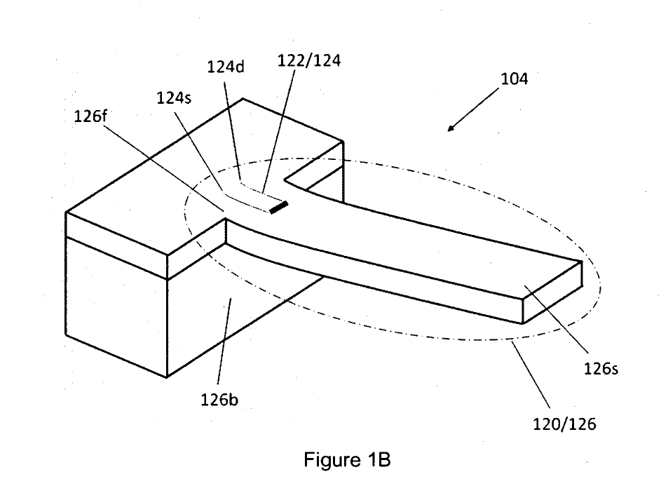

FIG. 2 shows a schematic of application specific integrated circuitry 200 for the active element arrangement present within both the substrate 102 and the housing structure 162 of the implantable device shown in FIG. 1A and FIG. 1C.

In various embodiments, the application specific integrated circuitry 200 may include the following blocks: a sensor interface block 202, a data converter block 204, a clock and control block 206, an energy recovery and power management block 208 and a data modulator block 210. The functionality of each block may be as follows.

The sensor interface block 202 may include an amplifier coupled to the sensor arrangement 104/first sensor 154/second sensor 155 to amplify and condition signals (associated with detected changes in the sensor arrangement 104) from the sensor arrangement 104 or to amplify and condition signals (associated with information on the difference between the first pressure detected by the first sensor 154 and the second pressure detected by the second sensor 155) from the first sensor 154 and the second sensor 155.

The data converter block 204 may include an analog to digital data converter coupled to the amplifier of the sensor interface block 202, the analog to digital data converter converting the output from the sensor arrangement 104/first sensor 154/second sensor 155 into digital data. Thus, the data converter block 204 may receive analog signals from the sensor interface block 202 and may convert it into digital format.

The data modulator block 210 may have a data modulator that is coupled to the analog to digital data converter of the data converter block 204 and the at least one inductive element 106, 108, 156 and 158. The data modulator may send the digital data to the at least one inductive element 106, 108, 156 and 158 for transmission. Thus, the data modulator block 210 may transmit digital data through the at least one inductive element 106, 108, 156 and 158 to an external reader module (such as the excitation source 110, see FIG. 1A or the excitation source 160, see FIG. 1C).

The clock and control block 206 may be coupled to the sensor interface block 202, the data converter block 204, the data modulator block 210 and the energy recovery and power management block 208. The clock and control block 206 may have a clock circuit coupled to synchronise the operation of the amplifier of the sensor interface block 202, the analog to digital data converter of the data converter block 204 and the data modulator of the data modulator block 210. Thus, the clock and control block 206 may supply clock and control signals to the rest of the blocks within the application specific integrated circuitry 200.

The energy recovery and power management block 208 may have an energy management circuit coupled to the at least one inductive element 106, 108, 156 and 158 to power the clock circuit of the clock and control block 206, the amplifier of the sensor interface block 202, the analog to digital data converter of the data converter block 204 and the data modulator of the data modulator block 210. Thus, the energy recovery and power management block 208 may recover energy from RF signals received through the at least one inductive element 106, 108, 156 and 158 and may power the application specific integrated circuitry 200.

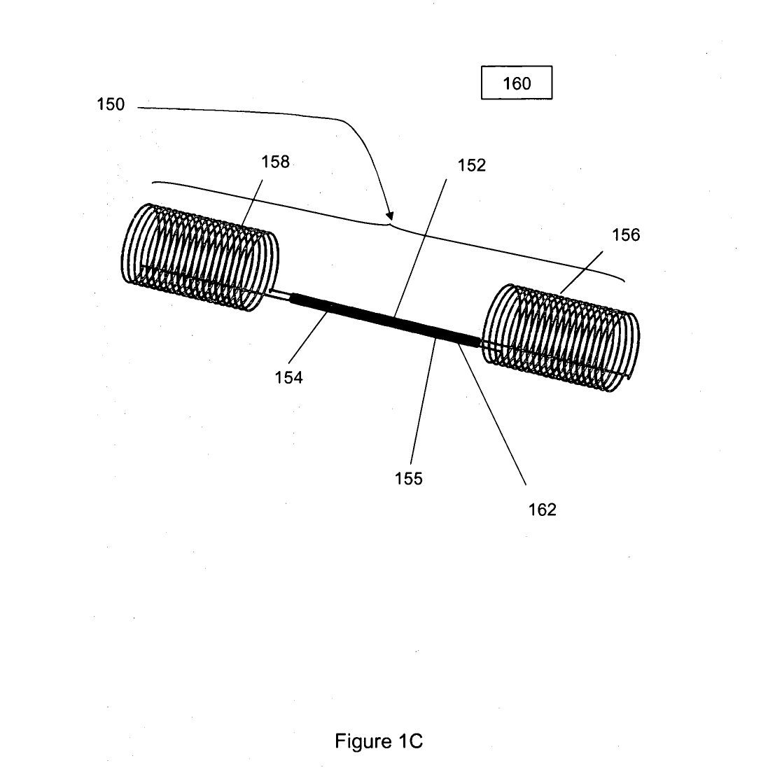

FIG. 3 shows a flow chart 300 illustrating a method of placing an implantable device, according to various embodiments, into the interior of a vessel.

At 302, a guide rod with an implantable device, in accordance with embodiments of the invention, is provided. At 304, the guide rod is inserted into the interior of a vessel. At 306, at least one inductive element of the implantable device is released to bias against the interior of the vessel so that the implantable device is secured to the interior of the vessel.

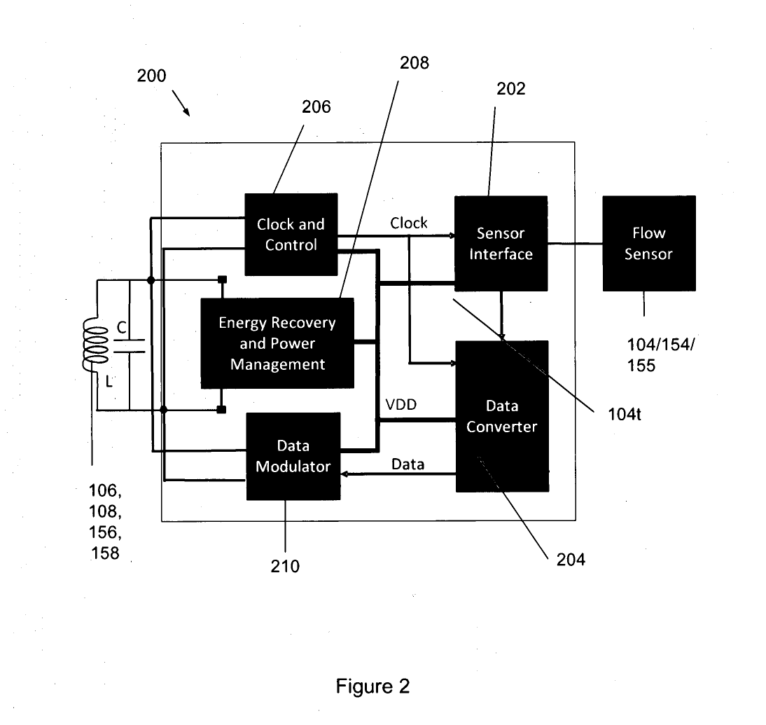

In one embodiment of the invention, the flow chart 300 may be implemented, as follows, with reference to FIGS. 4A to 4 C.

Prior to the anastomosis of a prosthetic graft 412, an implantable device 400, in accordance with various embodiments, will be placed and tested in-vitro, as shown in FIG. 4A. At least one inductive element provided as a coil 406, 408 is pre-compressed on a catheter 420 or guide rod. As an alternative to pre-compressing the coil 406, 408, shape memory alloy (SMA), such as nitinol, may be used.

Upon inserting the catheter 420 into the graft 412, the pre-compressed coil 406, 408 or SMA will be released, as shown in FIG. 4B, so that the coil 406, 408 will be anchored within the walls of the graft 412. The pre-compression may be engineered such that, after the pre-compressed coil 406, 408 is released, the coil 406, 408 will still be under stress and therefore bias against the interior walls of the graft 412, thereby firmly anchoring the implantable device 400 within the graft 412. Similarly, when SMA is used for the coil 406, 408, its expansion has to be such that the coil 406, 408 firmly grips to the interior walls of the prosthetic graft 412. In FIG. 4C, the catheter 420 is removed and the graft 412 is further taken for anastomosis. The same technique described with reference to FIGS. 3 and 4A to 4C may be used for deploying the implantable device 400 into vascular vessels (not shown) during by-pass conducts.

FIG. 5 shows a prosthetic vascular graft 502 that is implanted within an organism 504. The implantable device 100 (see FIG. 1A), 150 (see FIG. 1C) or 400 (see FIGS. 4A to 4C) may be introduced within the prosthetic vascular graft 502, using the technique described with reference to FIGS. 3 and 4A to 4C, prior to implantation of the prosthetic vascular graft 502 into the organism 504.

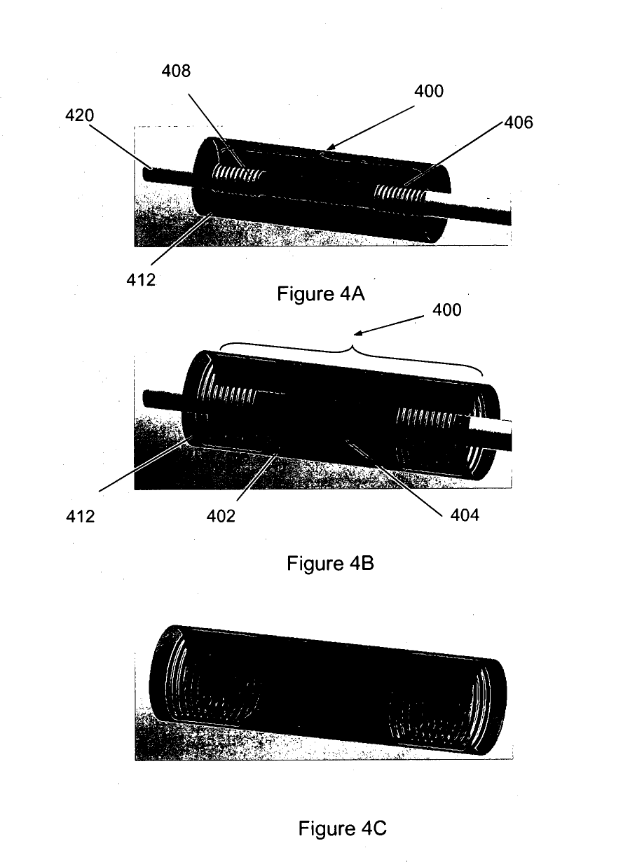

FIG. 6 shows a cross-sectional view of a portion 600 of the housing structure 162 of the implantable device 150 of FIG. 1C.

FIG. 6 shows that the sensors 154 and 155 are positioned along the direction 602 of blood flow, whereby the second sensor 155 is disposed downstream of the first sensor 154. In the embodiment shown in FIG. 6, both sensors 154 and 155 are embedded in a hermetically sealed substrate, where blood flows across over the surface of each membrane of the first sensor 154 and the second sensor 155. By measuring the difference between the pressure experienced at the first sensor 154 and the second sensor 155, the flow velocity may be deduced.

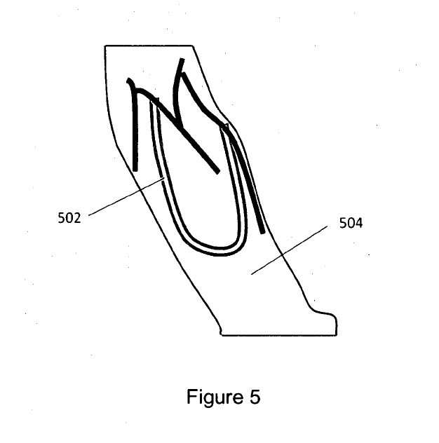

FIGS. 7A to 7F show cross-sectional views of the fabrication process to manufacture a sensor (such as the sensor arrangement 104 shown in FIG. 1A or the first sensor 154 and the second sensor 155 shown in FIG. 1C) of an implantable device, in accordance with various embodiments.

In FIG. 7A, a substrate 700 is provided. A buried oxide layer 702 (such as silicon dioxide) is formed on the substrate 700, followed by a semiconductor layer 704 (such as single crystal silicon). Thus, in the embodiment shown in FIG. 7A, the sensor may be fabricated using a silicon on insulator (SOI) wafer. However, it is also possible to perform fabrication using bulk silicon wafer, whereby poly-silicon may be used for the semiconductor layer 704.

The semiconductor layer 704 may be selectively etched, whereby the etched semiconductor layer 712 shown in FIG. 7B may be used to form nanowire structures 712a and 712b. Dopant, such as boron, is implanted (not shown) into the etched semiconductor layer 712 so that nanowire structures 712a and 712b formed from the etched semiconductor layer 712 are conductive, whereby the dopant concentration is controlled as a means to tune the piezoresistive properties of the nanowire structures 712a and 712b. The implantation is followed by oxidation of the structure 708. A SEM picture showing a top view of the structure 708 formed is shown on the right of FIG. 7B. From FIG. 7B, it will be appreciated that the structure 708 can be used to form a sensor using two silicon nanowires as the sensing element.

Another dopant, such as boron, is implanted (not shown) in a high dosage over the structure 708, except for the nanowire structures 712a and 712b, to form a low resistance ohmic contact at regions 712c and 712d of the etched semiconductor layer 712.

In FIG. 7C, a pre-metal dielectric layer (760a and 760b) is deposited and patterned to open the ohmic contacts 712c and 712d to expose portions 742a and 742b of the etched semiconductor layer 712. A metal layer is deposited (not shown) and patterned to form an interconnection, whereby FIG. 7C shows the result of the patterning of the metal layer to leave contact pads 710a and 710b.

Reinforcement trenches 762 (see SEM picture showing a top view of the structure of FIG. 7C) are created by etching into the substrate 700 and are subsequently filled with stiffening material such as silicon dioxide. The stiffening material is shaped by the reinforcement trenches to precisely define a cantilever anchor boundary during the silicon release step as a sidewall etch stop. In cases where precise definition of a cantilever anchor boundary is not required, the reinforcement trench etch may be skipped, but the stiffening material deposition may still needed for later formation of mechanical structures. The stiffening material extends past the reinforcement trench 762 to cover the whole wafer surface, including the pre-metal dielectric layer (760a and 760b) and the contact pads 710a and 710b. The stiffening material is then selectively etched to open the contact pads 710a and 710b, as shown in FIG. 7D. The etched stiffened structure 716 is shown in FIG. 7D.

An opening 774 (see FIG. 7E) is etched into the etched stiffened structure 716 to form the mechanical structure. The semiconductor substrate 700 is grinded down on the bottom side 700b of the semiconductor substrate 700, as shown in FIG. 7E, to facilitate release of the cantilever structure 720.

In FIG. 7F, the cantilever structure 720 release can be done from the top side 700t of the semiconductor substrate 700 using isotopic silicon etch or from the semiconductor substrate 700 bottom side 700b through anisotropic silicon etch.

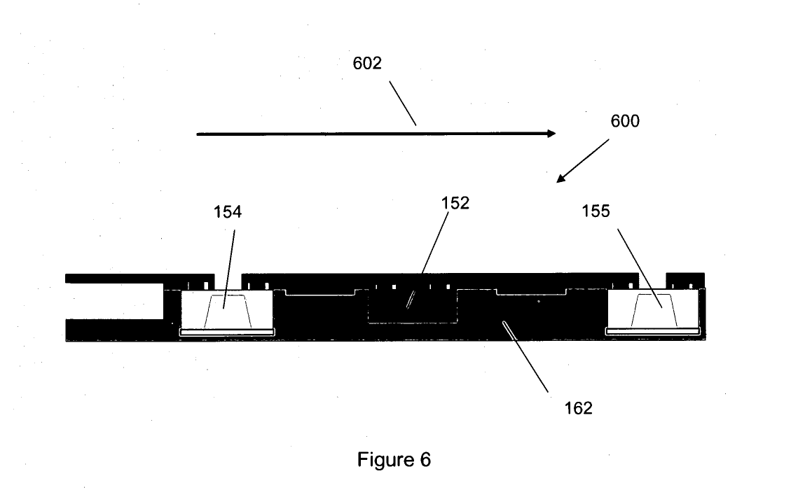

FIGS. 8A to 8C show cross-sectional views of an assembly process to produce an implantable device (such as the implantable device 150 shown in FIG. 1C) in accordance with various embodiments.

In FIG. 8A, the assembly process begins with the attachment of a first sensor 854, a second sensor 855 and an ASIC chip 852 onto a flexible interconnection cable 870, provided on a holding substrate 871, using a flip chip bonding technique.

In FIG. 8B, a housing structure 862 having openings 872 is provided. Adhesive 874 is selectively dispensed on a surface of the housing structure 862.

The structure 864 is positioned relative to the housing structure 862 such that each of the first sensor 854, the second sensor 855 and the ASIC chip 852 is aligned to insert into a respective opening 872. The flexible interconnection cable 870 is then bonded, via contact with the adhesive 874, to the housing structure 862 and cured to achieve hermetic sealing. The holding substrate 871 is removed to form the structure 876 shown in FIG. 8C.

Inductive coils (not shown) are attached to the housing structure 876 through conductive glue or soldering. The connection joints between the housing structure 862 and the coils are designed to be flexible to cater for the coil expansion during re-shaping.

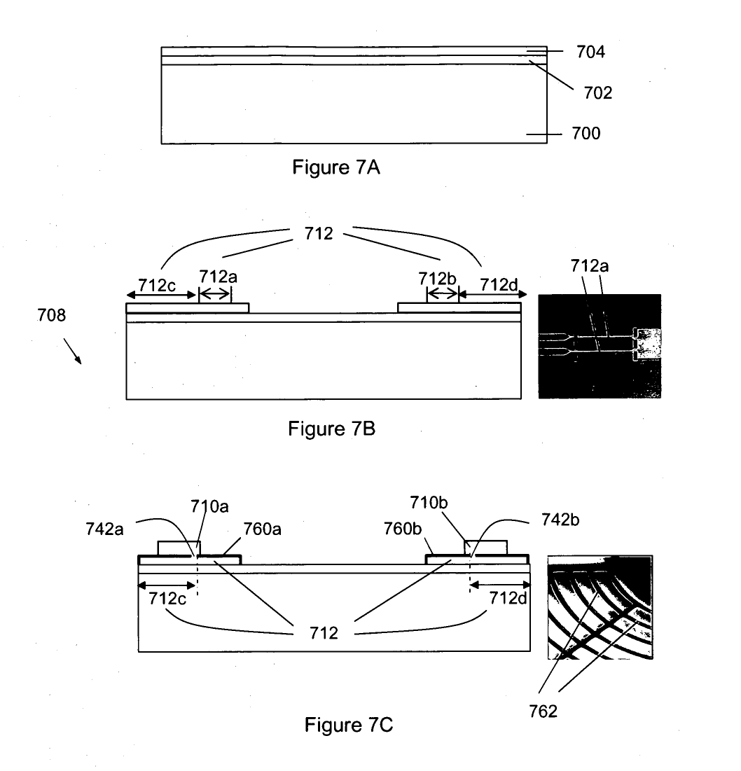

FIG. 9 shows chip architecture 900 for ASIC used by an implantable device according to various embodiments.

The architecture 900 includes a sensor interface block 902, a power management block 904, a rectifier/load modulator/limiter block 906, a clock extractor/ASK demodulator/power on reset (POR) block 908, a digital core block 910 and an ADC block 912.

The sensor interface block 902 is adapted to condition several input signals (Sensor 1, . . . , Sensor 4 and Sensor Ref.) from one or more sensors to which the ASIC is connected. The analog output signal from the sensor interface block 902 is then digitized by the ADC block 912 and converted to a serial bit stream. The sensor interface block 902 is also connected to the digital core block 910, the clock extractor/ASK demodulator/POR block 908 and the power management block 904. The digital core block 910 controls the sensor interface block 902 by an integration enable signal 914, an integration time control signal 916 and a gain control signal 918.

The power management block 904 is coupled to the sensor interface block 902, the digital core block 910 and the ADC block 912. The power management block 904 receives recovered DC power 920 from Rectifier/Load Modulator/Limiter 906 and sends a regulated DC supply to power the blocks 902, 908, 910 and 912.

The rectifier/load modulator/limiter block 906 is coupled to the digital core block 910; the clock extractor/ASK demodulator/POR block 908 and inductor coils 922. The rectifier/load modulator/limiter block 906 modulates data from transmission data signal 930 from the digital core block 910. The extracted data is transmitted through either of the coils 922.

The clock extractor/ASK demodulator/POR block 908 is also coupled to the digital core block 910 to provide demodulated received data 924, a clock signal 926 and a POR signal 928 to the digital core block 910. The clock extractor/ASK demodulator/POR block 908 demodulates the received signal from either of the coils 922 and sends the demodulated data via the received data signal 924.

The digital core block 910 serves as the processor for the chip architecture 900. The digital core block 910 is coupled to the ADC block 912.

Additional key features of the architecture 900 are as follows.

DC power recovering blocks include a rectifier (from the rectifier/load modulator/limiter block 906) with a parallel resonant tank at the input, a limiter (from the rectifier/load modulator/limiter block 906) and the power management block 904.

In a vascular prosthetic graft, very little RF energy at 13.56 MHz reaches an implanted ASIC, after skin and tissue absorption, for the RF-to-DC energy conversion to power the ASIC. Hence, increasing the efficiency of the rectifier and reducing the power consumption of the ASIC is critical. The chip architecture 900 achieves this by providing a parallel resonant LC tank having an optimum quality factor and a highly efficient rectifier designed along with low dropout (LDO) regulators.

The resistance of nanowire sensors changes in proportion to flow rate. The sensor interface block 902 converts the resistance to analog voltage. The analog voltage is in turn converted to digital data by, for example a 10-bit ADC in the ADC block 912. A clock signal is extracted from an incoming carrier from an external hand-held device, which may have a carrier frequency f_{c} of 13.56 MHz. The sampling clock for the ADC block 912 may be 106 kHz which is f_{c}/128. The external device configures the implanted ASIC by sending a command. After selecting the sensor to be read and setting the parameters such as gain, integration time, etc., the ADC block 912 clock is generated. The sensor data is digitized by the ADC block 912 and converted to a serial bit stream. The digital data is coded to a desired format in the digital core 910 and sent to the external device by backscattering the incoming RF carrier through load modulation.

Unregulated DC voltage from a rectifier in the rectifier/load modulator/limiter block 906 is regulated by low-power LDO voltage regulators. The power management block 904 generates desired reference voltages for the sensor interface block 902 and a SAR ADC in the ADC block 912.

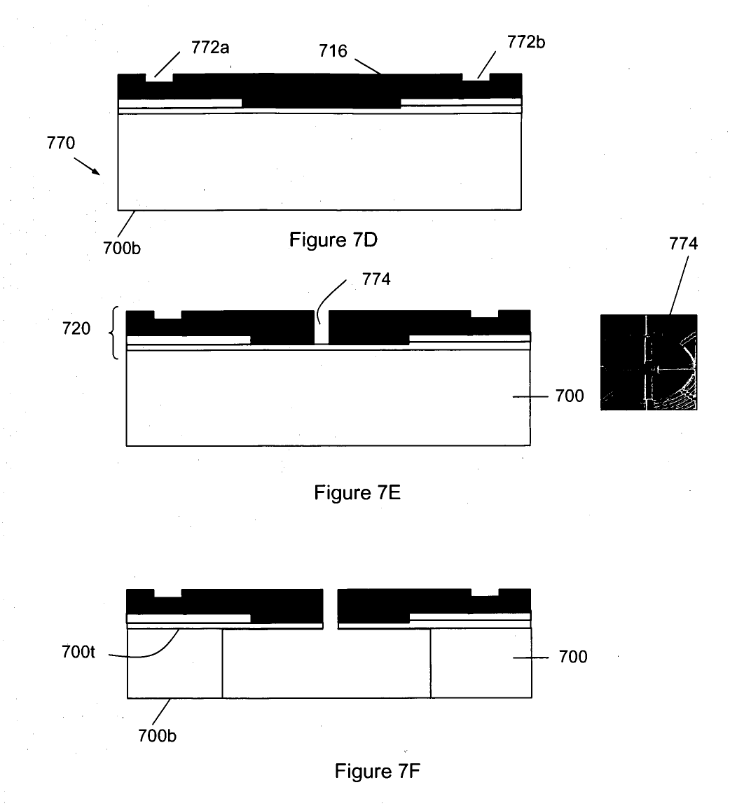

FIG. 10 shows a schematic diagram of an RF front-end 1000 of an ASIC used by an implantable device according to various embodiments. The RF front-end 1000 connects to an inductor coil 1010.

The RF front-end 1000 includes a rectifier stage 1002, a clock extractor stage 1004, an ASK demodulator stage 1006 and a backscatter modulator stage 1008.

Both the rectifier stage 1002 and the backscatter modulator stage 1008 are coupled to the ASK demodulator stage 1006 and an inductor coil 1010. The ASK demodulator stage 1006 is coupled to the clock extractor stage 1004.

The power conversion efficiency (PCE) of the rectifier stage 1002 is an important parameter. For converting AC energy to DC energy, an eight-stage 1002b differential-drive rectifier is used. The rectifier core 1002a includes transistors 1012 connected in a cross-coupled bridge configuration. A differential-drive active gate bias mechanism 1014 enables to achieve both low ON-resistance and small reverse leakage of diode-connected MOS transistors 1012 at the same time, resulting in a high PCE. Each stage 1002b is serially stacked along the DC path and connected in parallel to the input RF terminals 1016 and 1018. By using this multi-stage configuration, appropriate DC output voltage is obtained at the optimal operating point where the PCE is maximized.

The clock extractor stage 1004 includes an input AC-coupled amplifier 1004b and a Schmitt trigger 1004a. The clock signal is divided by two at 1004c, buffered at 1004d and fed 1004e to a digital core as its reference clock.

The ASK demodulator stage 1006 includes a diode-connected transistor arrangement 1006a for envelope detection, an averaging circuit 1006b, a comparator 1006c and a buffer 1006d. At the ASK demodulator stage 1006, the envelope of the received ASK-modulated signal 1006e is compared, at the comparator 1006c with the average value of the envelope of the signal 1006f from the averaging circuit 1006b to obtain a command from an external device.

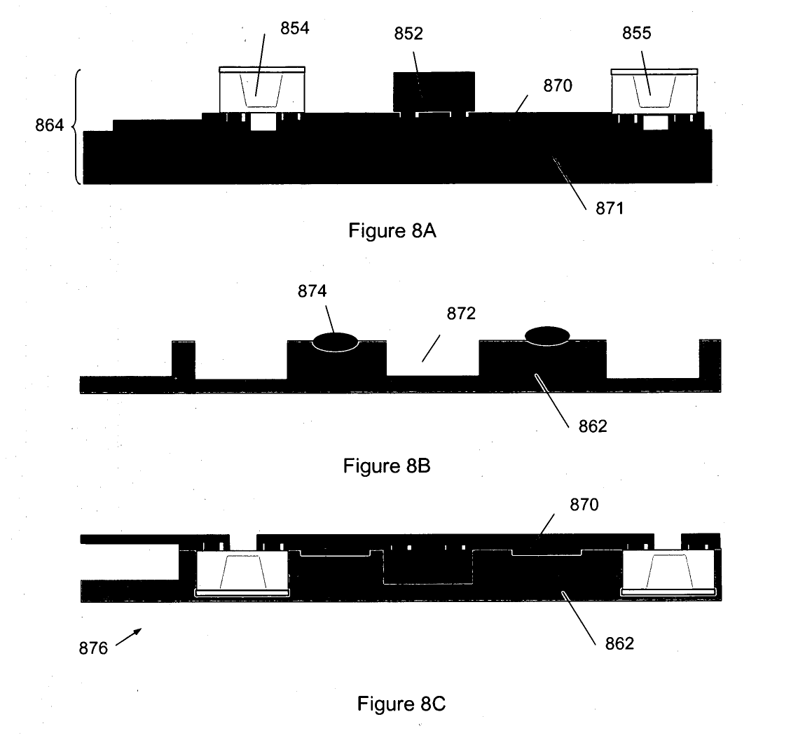

FIG. 11 shows a flow chart 1100 implemented in a digital baseband and controller of an ASIC used by an implantable device according to various embodiments.

At 1102, the digital baseband and controller is inactive or in a power off mode. At 1104, when inductively powered by an external device, the digital baseband and controller enters into a receive mode. At 1106, the digital baseband and controller will determine whether the external device is transmitting a SOF (start of frame). If a SOF is not received, the digital baseband and controller remains in its receive mode at 1104. If the SOF is received, the digital baseband and controller then determines an implant ID, at 1108, of the external device. At 1110, the digital baseband and controller checks whether the implant ID of the external device matches the ID of the ASIC. If there is no match, the digital baseband and controller returns to its power off mode at 1102. If there is a match, the digital baseband and controller then proceeds to 1112 to receive data from a flow sensor of the implantable device regarding variation in fluid flow rate. At 1114, the digital baseband and controller enters into an ADC mode to digitize the data providing information on the variation in the fluid flow rate. At 1116, the digital baseband and controller transmits the information on the variation in the fluid flow. The digital baseband and controller then returns to its power off mode at 1102.

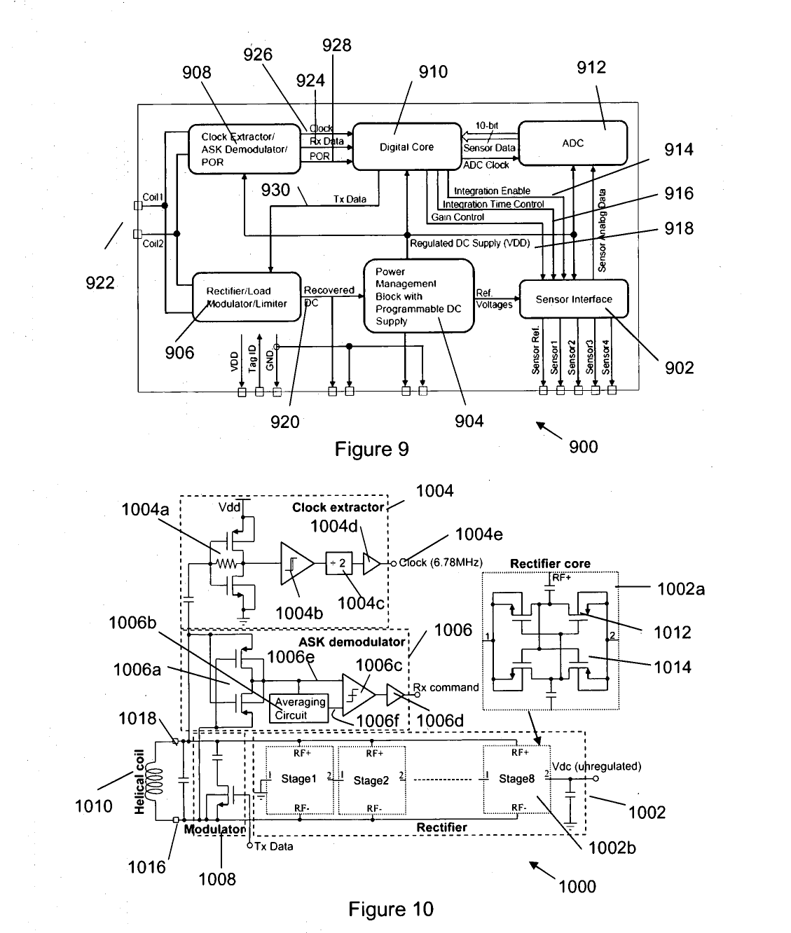

FIG. 12A shows a schematic diagram of a sensor interface circuit 1200 of an ASIC used by an implantable device according to various embodiments. The sensor interface circuit 1200 may be compatible with nanowire-based piezoresistive sensors used to sense fluid flow, where the sensors resistances change according to pressure applied to the sensors. The change in resistance, ΔR, can be in the range of ±10% to ±30%. The sensor interface circuit 1200 converts ΔR, into analog voltage.

The sensor interface circuit 1200 includes a switched current integrator stage 1202 coupled to a single-ended to differential gain stage 1204. Operation of the sensor interface circuit 1200 is explained with refererence to FIG. 12B, which shows a timing diagram and output voltage waveform of the switched current integrator stage 1202.

During reset (RST) period 1208, switch S1_{RST} and S2_{RST} of the switched current integrator stage 1202 are closed while switch S_{INT} is open, making op-amp 1206 in the unity gain configuration. The offset of the op-amp 1206 is stored in capacitor C_{OFF} during the period 1208. A non-overlapping time, pre-integration hold 1212, between the reset period 1208 and the integration period 1210 prevents shorting of a sensor (such as the piezoresistive sensor) to ground before S2_{RST} is fully opened. During the integration (INT) period 1210, S1_{RST} and S2_{RST} are opened while switch S_{INT} is closed. A selected channel sensor current is then integrated through capacitor C_{INT}. During the period 1210, a voltage of 100 mV is applied across the sensor. The output voltage of the integrator, Voutᵢₙₜ, settles at a voltage level that depends on both the integration period 1210 (which is programmable) and the sensor resistance.

The single ended output voltage Vout_{INT} from the switched current integrator stage 1202 is amplified and converted to a differential signal by the single-ended to differential gain stage 1204. The single-ended to differential gain stage 1204 includes a fully differential folded-cascade op-amp with a switched-capacitor common-mode feedback (SC-CMFB) circuit 1214, a switched-capacitor (SC) feedback circuit 1216/1218. The gain of the single-ended to differential gain stage 1204 is equal to C1/C2 and can be controlled as C1 is a 3-bit programmable capacitor bank.

The operation of the single-ended to differential gain stage 1204 is as follows. When S1 is closed, the input voltage is stored in the capacitor C1. The op-amp 1214 holds the previous value while the charge at C2 is reset during this period. When S2 is closed, the charge in C1 is transferred to C2. The cycle repeats again. Capacitor C3 keeps the op-amp 1214 in a closed-loop and holds the previous voltage. However, capacitor C3 does not contribute to the gain of the single-ended to differential gain stage 1204, which (as earlier mentioned) is given by C1/C2.

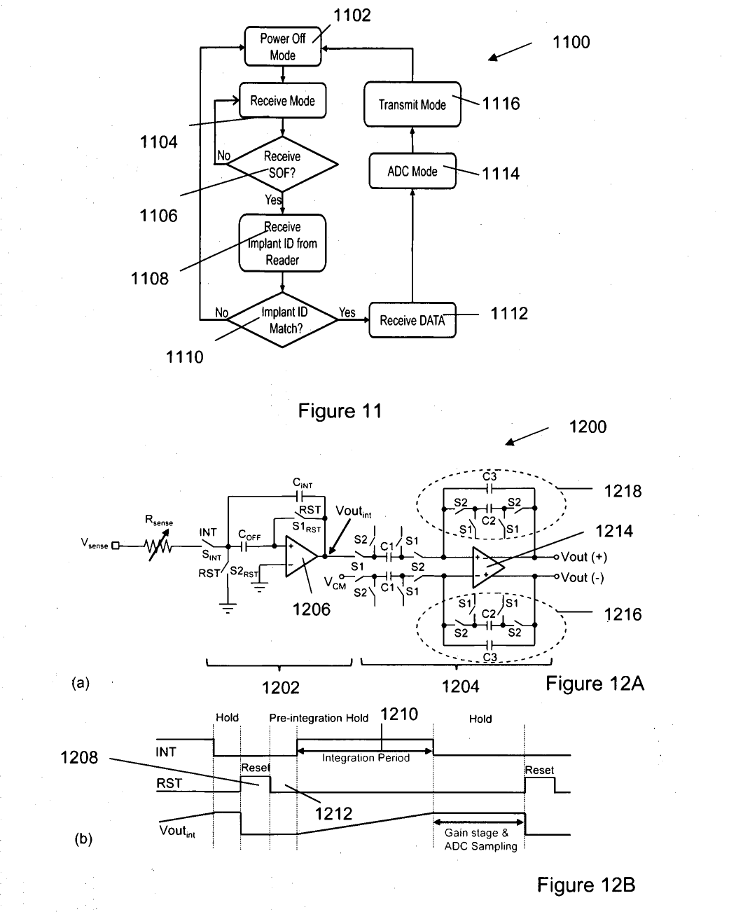

FIG. 13 shows a schematic diagram of a SAR ADC 1300 of an ASIC used by an implantable device according to various embodiments. The SAR ADC 1300 is a suitable solution for micro-power medical devices due to their low power consumption.

The SAR ADC 1300 includes a capacitor array 1304a and 1304b, a switching array 1306a and 1306b, a time-domain comparator 1308 and switching logic 1310. A non-binary redundant algorithm is applied to the capacitor array 1304a and 1304b of the SAR ADC 1300. The time-domain comparator 1308, utilized to reduce the power consumption, converts the voltage signal to pulse width and compares the duration of the pulses.

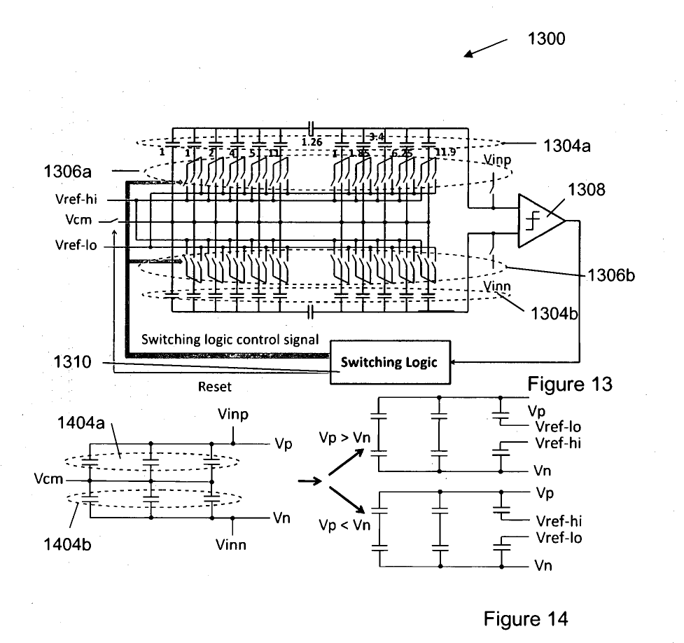

A common-mode resetting tri-level switching scheme is applied to the SAR ADC, as shown in FIG. 14. During the sampling period, the common-mode resetting tri-level switching scheme samples the input signal onto the top plates of the capacitor array 1404a and 1404b (equivalent to the capacitor arrays 1304a and 1304b in FIG. 13), while the bottom plates are reset to V_{cm} which is equal to V_{ref}/2. By doing so, the 1^{st} MSB can be determined during the sampling period without a need for another cycle for the MSB decision. As a result, one conversion cycle is saved. Based on previous bit decisions, the bottom plates of the following capacitor pairs will be switched to either V_{ref-hi} or V_{ref-lo} whereas the rest of the differential capacitors are connected to each other, creating a virtual V_{cm} (=V_{ref}/2). Utilizing the tri-level switching scheme, an N-bit SAR ADC with M redundant bits requires N+M capacitors, and takes only N+M cycles to complete the conversion. Top-plate sampling may be subject to charge injection, but using a fully differential structure and complementary switches can reduce the effect. In an integrated system, an additional voltage level would mean an additional buffer needed in the system to hold the voltage. Such low impedance buffer will significantly increase the overall power consumption of the whole system. In order to avoid using a third voltage level, the reference voltage can be designed in such a way that V_{ref-lo} is 0V, and V_{ref-hi} is set to V_{ref}, which is selected to be equal to the input common mode voltage (Vᵢₙ_{_{—}}_{common}).

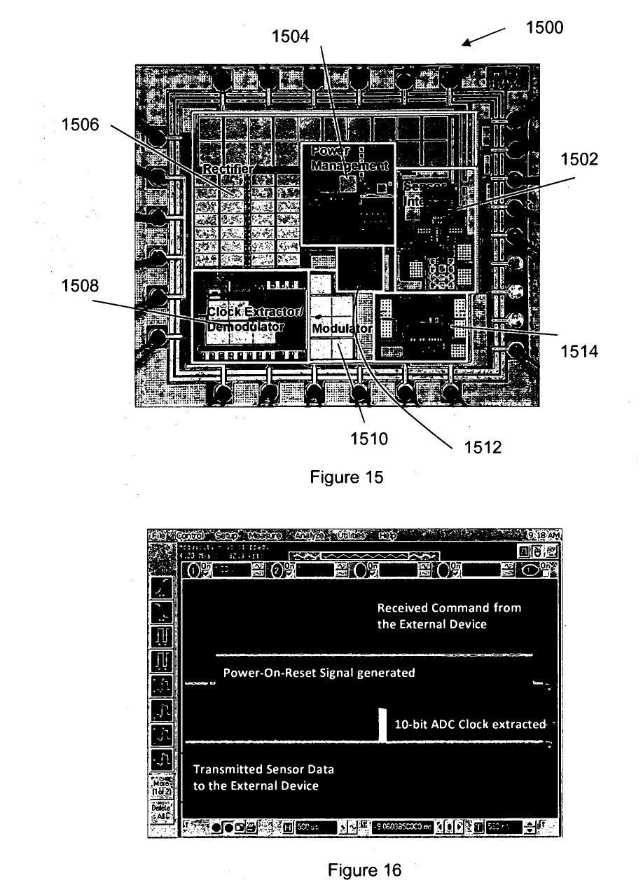

FIG. 15 shows a microphotograph of an ASIC chip 1500 used by an implantable device according to various embodiments. The ASIC chip 1500 has been fabricated in 0.18 μm CMOS process. The ASIC chip 1500 occupies a total active area of 1.5×1.78mm² and consumes a total power of 21.6 μW.

The ASIC chip 1500 includes a sensor interface block 1502, a power management block 1504, a rectifier block 1506, a clock extractor/demodulator block 1508, a load modulator block 1510, a digital core block 1512 and an ADC block 1514.

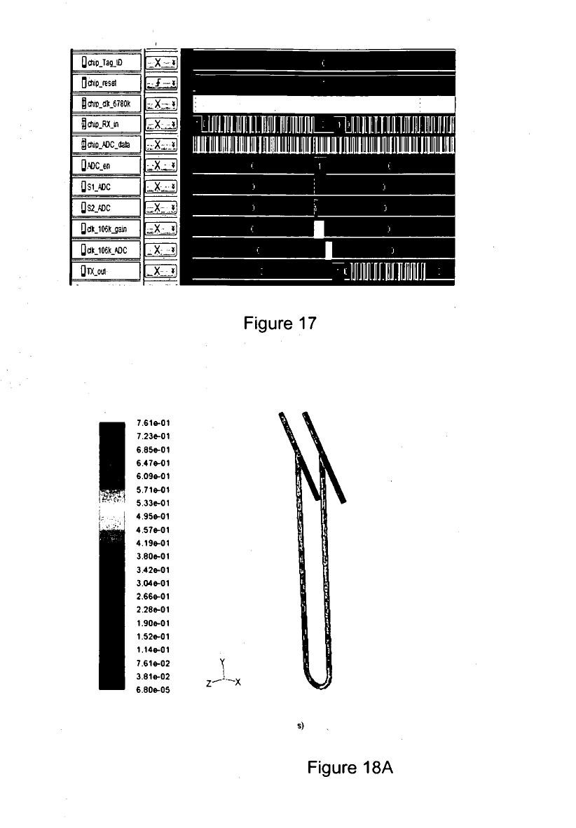

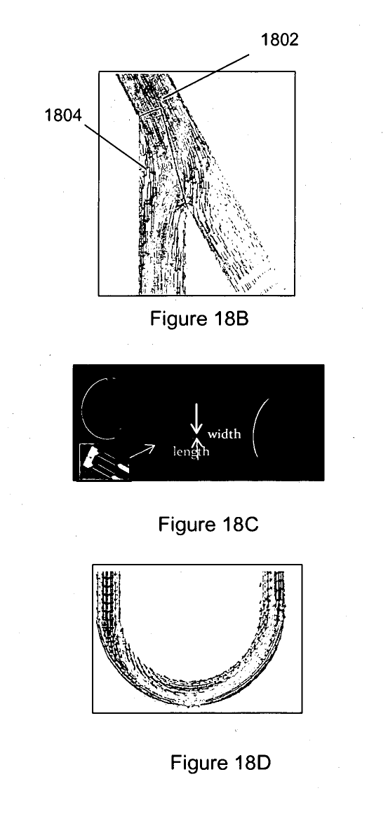

As shown in FIG. 16, RF power from a reader was converted to DC supply to power the ASIC chip 1500 and command from a reader is demodulated, clock is extracted from the incoming carrier, power-on-reset signal is generated to reset the digital baseband, clock for the ADC block 1514 is generated to convert analog sensor information to digital in 10-bit ADC and this data is processed in digital baseband and fed to the load modulator block 1510 to backscatter the unmodulated carrier from the external device. Measured timing diagrams are shown in FIG. 17. The measured ASIC chip 1500 performance is summarized in Table I.

TABLE 1
Measured Performance Summary of ASIC chip

| Parameter | Measured Result |
|---|---|
| Carrier frequency | 13.56 MHz |
| Modulation and Demodulation | ASK (programmable modulation depth |
| (Both External and Implant | from 10% to 90% in steps of 10%) |
| Devices) | |
| Communication Protocol | Modified and simplified from ISO |
| | 14443 RFID standard |
| Rectifier efficiency | 66% |
| Power management block | Efficiency: 56%; |
| | Power consumption: 12.8 μW |
| ADC | Resolution: 10 bits |
| | ENOB: 8.6 bits @ 5 kHz input |
| | 7.4 bits @ 25 kHz input |
| | INL/DNL: ±1.5LSB/±0.6LSB |
| Total power consumption | 21.6 μW |
| | (RF front-end: 5 μW; ADC: 0.4 μW; |
| | Sensor interface: 1.4 μW; |
| | Power management: 12.8 μW; |
| | Digital core: 2 μW) |

Notwithstanding the materials, along with their respective parameters, presented thus far to fabricate an implantable device using methods in accordance to embodiments of the invention, an implantable device built in accordance to the invention may be composed of the following materials and have the following respective parameters.

The implantable device may be made of biocompatible packaging.

The sensor arrangement (104, 404) may be formed of poly silicon, single crystal silicon, silicon oxide or nitride and may have the following dimensions: 0.5 mm×0.5 mm×0.5 mm, with a tolerance of ±0.1 mm per dimension. Detection of compressive and tensile forces occurs preferably along one axis for the sensor arrangement (104, 404), to sense fluid flow having velocity of 40 to 60 cm/s, with a tolerance of ±1 cm/s and 4 cm/s resolution. The sensors (154, 155) may also be formed of poly silicon single crystal silicon, silicon oxide or nitride.

The at least one inductive element (106, 108, 156, 158, 406, 408) may be formed of resilient material providing anchorage when the inductive element (106, 108, 156, 158, 406, 408) is deformed. The at least one inductive element (106, 108, 156, 158, 406, 408) may be formed of nitinol or titanium. The at least one inductive element (106, 108, 156, 158, 406, 408) preferably provides for high-efficiency inductive coupling for power and data transfer @13.56 MHz.

The substrate (102, 402) may be formed of bulk silicon or silicon on insulator. The housing structure 162 may be formed by silicon, silicon oxide or polymer. The ASIC provided in the substrate (102, 402) or the housing structure 162 preferably operates at low power levels of around <10 uW.

### Experimental Data

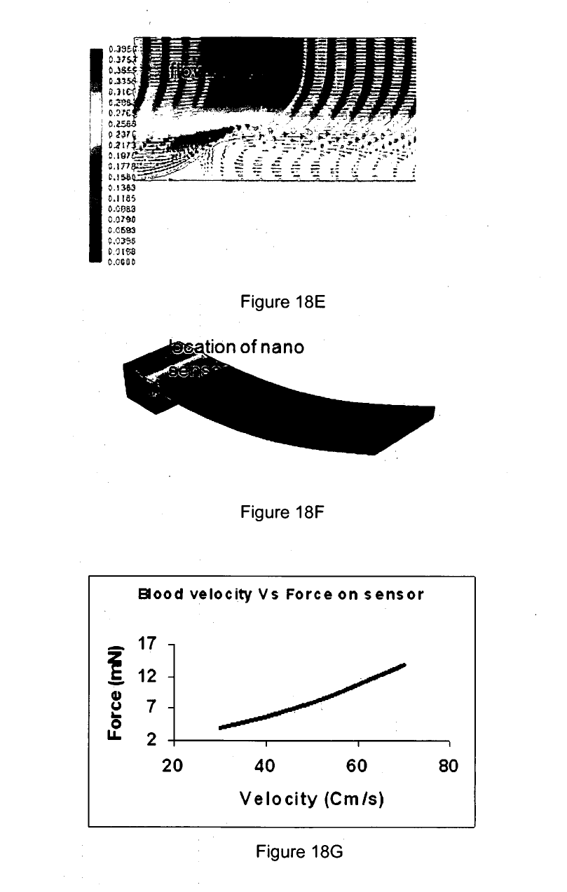

Simulation of an implantable device according to an embodiment having dimensions of 500 um×500 um, was performed under laminar fluidic (blood) flow conditions inside a prosthetic vascular graft shown in FIGS. 18A to 18D for conducting experiments for detecting fluid velocity between 10 to 60 cm/s.

In the experiment, a prosthetic graft was fused at each opposing end to a respective blood vessel, as shown in FIG. 18A. FIG. 18B shows the connection between one end of the prosthetic graft 1804 and the blood vessel 1802. The implantable device was located at least 6 cm away from the start point of anastomosis (proximal). Silicon nanowires (SiNW) were used as the sensing elements of the implantable device. The SiNW had a gauge factor around 600, a length of 80 um, a width of 5 um and was able to deflect up to 15 um. FIG. 18C shows a section of the prosthetic vascular graft where the implantable device is located and that a healthy prosthetic graft was used. According to the results, the blood flow offered drag force on the walls where the implantable device was placed and the drag force deformed the movable structure in the implantable device. The deformation offered change in the strain and the nanowire was located exactly where the strain is high. The strain changed the resistance of the nanowire, which was detected by the ASIC. The drag force offered for various flow velocities of blood was computed and shown in FIG. 18G. From FIGS. 18E and 18F, it was noted that when the fluid velocity increased, deformation of the sensor increased.

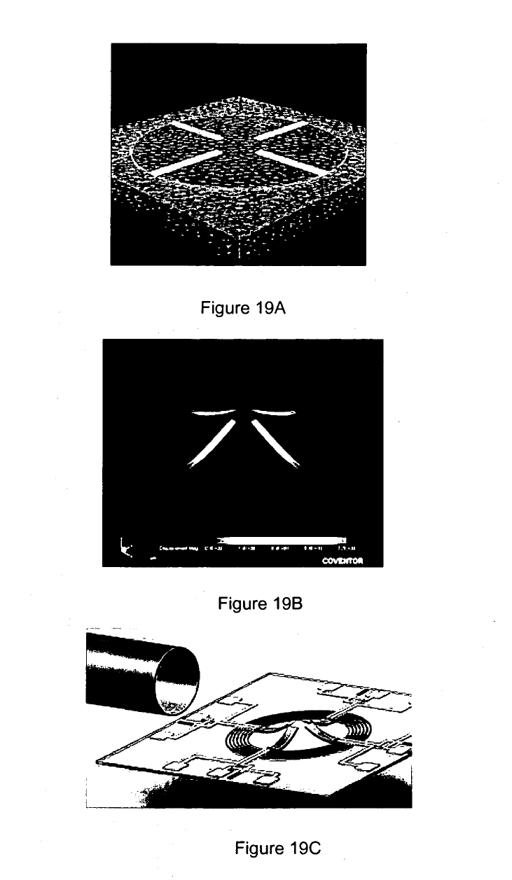

An experiment was performed on a fabricated sensor, shown in FIG. 19A, to check the functionality of the sensor for the drag force. Air was blown at different velocities, where a cantilever deformed (as shown in FIG. 19B) in the direction of the air flow. It was found that current in the nanowire changed with change in the air velocity, thereby exhibiting a trend similar to that discover in the simulation results discussed above. FIG. 19C shows a schematic of the experiment conducted. From the experiment, it was found that at higher velocities, drag force was dominated by lift force due to air being bounced back, thus causing localized turbulence and lifting the cantilever instead of drag. Results of the sensor response was plotted and shown in FIG. 19D.

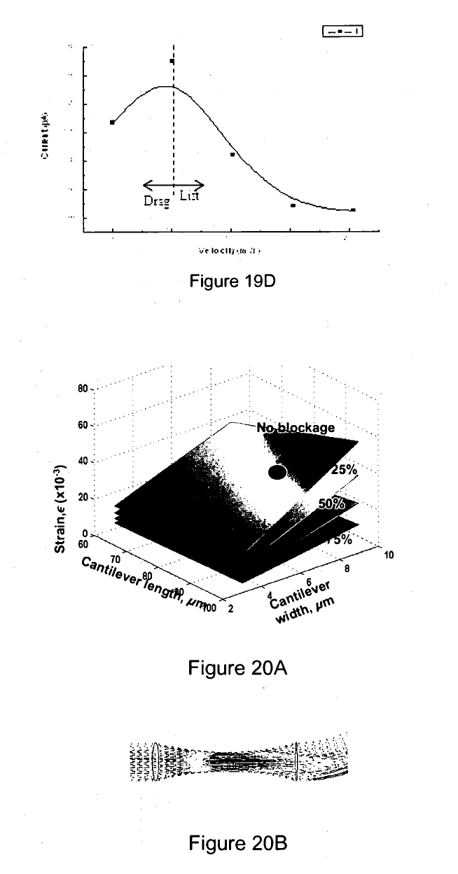

FIG. 20A shows the parametric study of the strains obtained for various blockage percentages for the varying parameters such as cantilever width and length for sensors in a graft having no blockage, 25% blockage, 50% blockage and 75% blockage. As an illustrative example, FIG. 20B shows a stenoses graft having 50% blockage. 50% blockage is a point where surgeons start checking flow rate more often and change medication and prepare for surgical procedures.

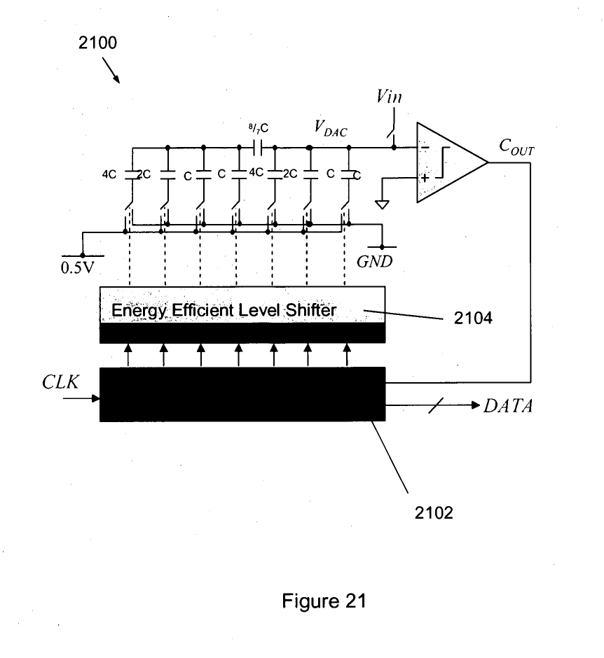

FIG. 21 shows a schematic of an 8-bit successive-approximation ADC register (SAR) 2100 with sub-threshold control logic, able to meet ultra low power requirements of an implantable device according to embodiments of the present invention. The ADC 2100 was provided inside the ASIC of the implantable device. The SAR and control logic block 2102 operates at around 0.5V, while the level shifter voltage 2104 operates at around 1.2V. The ADC 2100, fabricated using 0.18 um technology, consumed about 2.49 uW of power and was able to convert data at around 150 kS and produced around 8.9 effective number of bits.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. An implantable device for detecting variation in fluid flow rate, the implantable device comprising:
a substrate having an active element arrangement;
a sensor arrangement having a first portion that is mechanically secured and a second portion that is freely deflectable, the sensor arrangement in electrical communication with the active element arrangement, wherein the active element arrangement is configured to detect changes in deformation of the sensor arrangement and produce an output in response to the detected changes; and
at least one inductive element mechanically coupled to the substrate and in electrical communication with the active element arrangement, wherein the inductive element is adapted to power the active element arrangement through inductive coupling to an excitation source, wherein the inductive element is adapted to transmit the output associated with the detected changes in the sensor, and wherein the inductive element is adapted to provide a biasing engagement in contact with an interior surface to which the implantable device is attached.

2. The implantable device of claim 1, wherein the active element arrangement comprises application specific integrated circuitry.

3. The implantable device of claim 2, wherein the application specific integrated circuitry comprises an amplifier coupled to the sensor arrangement.

4. The implantable device of claim 3, wherein the application specific integrated circuitry further comprises an analog to digital data converter coupled to the amplifier, the analog to digital data converter converting the output associated with the detected changes in the sensor arrangement into digital data.

5. The implantable device of claim 4, wherein the application specific integrated circuitry further comprises a data modulator coupled to the analog to digital data converter and the at least one inductive element, the data modulator sending the digital data to the at least one inductive element for transmission.

6. The implantable device of claim 5, wherein the application specific integrated circuitry further comprises a clock unit coupled to synchronize the operation of the amplifier, the analog to digital data converter and the data modulator.

7. The implantable device of claim 5, wherein the application specific integrated circuitry further comprises an energy management unit coupled to the at least one inductive element, to power the clock unit, the amplifier, the analog to digital data converter and the data modulator.

8. The implantable device of claim 1, wherein the first portion of the sensor arrangement is mechanically coupled to the substrate.

9. The implantable device of claim 1, wherein the first portion of the sensor arrangement is mechanically coupled to the at least one inductive element.

10. The implantable device of claim 1, wherein the sensor arrangement is a nano electromechanical structure or a micro electromechanical structure.

11. (canceled)

12. The implantable device of claim 1, wherein the sensor arrangement comprises a movable structure; and at least one sensing element formed on or within the movable structure.

13. The implantable device of claim 12, wherein the movable structure comprises a cantilever or diaphragm.

14. (canceled)

15. The implantable device of claim 1, wherein the sensor arrangement is disposed between the at least one inductive element and a further element formed of resilient material providing anchorage when deformed.

16. (canceled)

17. The implantable device of claim 1, wherein the at least one inductive element is formed of resilient material providing anchorage when the inductive element is deformed.

18-20. (canceled)

21. An implantable device for detecting variation in fluid flow rate, the implantable device comprising:
a first sensor configured to detect a first pressure;
a second sensor disposed downstream of the first sensor and configured to detect a second pressure;
a housing structure having an active element arrangement, both the first sensor and the second sensor being in electrical communication with the active element arrangement, wherein the active element arrangement is configured to obtain the first pressure, the second pressure and produce an output containing information on the difference between the first pressure and the second pressure; and
at least one inductive element mechanically coupled to the housing structure and in electrical communication with the active element arrangement, wherein the inductive element is adapted to power the active element arrangement through inductive coupling to an excitation source, wherein the inductive element is adapted to transmit the output from the active element arrangement and wherein the inductive element is adapted to provide a biasing engagement in contact with an interior surface to which the implantable device is attached.

22. The implantable device of claim 21, wherein the active element arrangement comprises application specific integrated circuitry.

23. The implantable device of claim 22, wherein the application specific integrated circuitry comprises a sensor interface coupled to the first sensor or the second sensor.

24-32. (canceled)

33. The implantable device of claim 21, wherein the first sensor and the second sensor are disposed between the at least one inductive element and a further element formed of resilient material providing anchorage when deformed.

34. The implantable device of claim 21, wherein the at least one inductive element is formed of resilient material providing anchorage when the inductive element is deformed.

35-40. (canceled)

41. A method of placing an implantable device into the interior of a vessel, the method comprising:
providing a guide rod with the implantable device of any one of the preceding claims mounted;
inserting the guide rod into the interior of a vessel; and
releasing the at least one inductive element of the implantable device to bias against the interior of the vessel so that the implantable device is secured to the interior of the vessel.
